(19) United States
(12) Patent Application Publication
(10) Pub. No.: US 2002/0127609 A1
(43) Pub. Date: Sep. 12, 2002

(54) Screening for enzyme stereoselectivity utilizing mass spectrometry

(75) Inventors: S. Christopher Davis, San Francisco, CA (US); Yong Hong Chen, Foster City, CA (US); Lorraine J. Giver, Santa Clara, CA (US); Kurt Vogel, Madison, WI (US)

Correspondence Address: QUINE INTELLECTUAL PROPERTY LAW GROUP, P.C., P O BOX 458, ALAMEDA, CA, 94501, US

(73) Assignee: Maxygen, Inc., Redwood City, CA

(21) Appl. No.: 10/082,780

(22) Filed: Feb. 22, 2002

Related U.S. Application Data
(60) Provisional application No. 60/271,120, filed on Feb. 23, 2001.
(60) Provisional application No. 60/278,934, filed on Mar. 26, 2001.

(51) Int. Cl. G01N 33/53

(52) U.S. Cl. 435/7.1

(57) ABSTRACT

Methods of screening for enzyme stereoselectivity that include detecting isotopically labeled products by mass spectrometry are provided. The methods are particularly suitable for screening enzyme libraries for stereoselectivity.

### REFERENCE TO RELATED APPLICATIONS

This application claims the benefit of U.S. Provisional Application No. 60/271,120, filed Feb. 23, 2001, and U.S. Provisional Application No. 60/278,934, filed Mar. 26, 2001, both of which are incorporated by reference in their entirety.

### COPYRIGHT NOTIFICATION

Pursuant to 37 C.F.R. § 1.71(e), Applicants note that a portion of this disclosure contains material which is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### BACKGROUND OF THE INVENTION

Asymmetric transformations include the conversion of a racemate into a pure enantiomer or into a mixture in which one enantiomer is present in excess, or of a diastereoisomeric mixture into a single diastereomer or into a mixture in which one diastereoisomer predominates. Enzymes such as lipases that catalyze asymmetric transformations are of great interest for the production of fine chemicals and intermediates, food products and supplements, and for other uses.

The throughput of many screening techniques currently used for the discovery of, e.g., selective lipases from expression libraries is generally limited, because the screens typically involve assaying enzymes for activity against single purified compounds. As a consequence, these screens also do not provide direct measurements of enzyme enantioselectivity or diastereoselectivity in the presence of multiple substrate molecules. In addition, the sensitivity of certain existing screening techniques is also limited, because these screens typically rely on detecting remaining starting materials following screening reactions, rather than detecting reaction products directly.

In general, enhanced throughput methods of screening expression libraries for desired properties would be desirable. The present invention provides new methods of screening for enzyme stereoselectivity and detecting isotopically labeled reaction products by mass spectrometry. These and a variety of additional features will be apparent upon complete review of the following.

### SUMMARY OF THE INVENTION

The present invention generally relates to screening enzymes for desired traits or properties. In particular, the invention provides methods of screening for enzyme stereoselectivity. The methods include simultaneously screening an enzyme for activity towards multiple substrate molecules, which are typically pseudo-stereoisomers, to provide a direct measurement of enzyme selectivity upon mass spectrometric detection and quantification of products. The methods also include screening for enzyme stereoselectivity in reactions that involve pseudo-meso compounds. Advantages of the invention include improved screening sensitivities due to the detection of reaction products, rather than remaining substrate molecules in a given mixture or other reaction medium. Detection limits are also enhanced relative to certain existing methods owing to the minimum constitution of quantified products, which provide for improved discrimination over smaller background molecules. Furthermore, the screening methods of the invention typically provide a measure of initial reaction kinetics (i.e., at low conversions).

In one aspect, the invention is directed to a method of screening for enzyme stereoselectivity that includes providing a plurality of substrate molecules of one or more substrate molecule types. The substrate molecule types include one or more leaving groups in which at least one of the one or more leaving groups of at least one of the one or more substrate molecule types includes at least one isotopic label. The methods also include contacting an enzyme (e.g., a hydrolase, such as a lipase, an esterase, a protease or the like) with the plurality of substrate molecules of the one or more substrate molecule types. The enzyme converts one or more of the substrate molecules to two or more products in which at least one of the two or more products includes the at least one isotopic label. In addition, the method includes quantifying the two or more products mass spectrometrically to screen for enzyme stereoselectivity. Typically, the product(s) are detected when conversion of substrate(s) to product(s) is low. In preferred embodiments, one or more of the two or more products have three or more carbon atoms, e.g., to improve the detection limit of the mass spectrometric detection and quantification relative to the detection and quantification of products having fewer carbon atoms, such as acetyl moieties. That is, detection of, e.g., propyl, butyl, or larger moieties provide for enhanced discrimination over, e.g., small molecule organic contaminants, such as other components of cells and media that typically have masses that are similar to products with fewer carbon atoms. Thus, generally, the substrate leaving groups typically have three or more carbon atoms. In certain embodiments, the substrate leaving groups, and hence the products, have four or more carbon atoms.

In some embodiments, the plurality of molecules of the one or more substrate molecule types include a mixture (e.g., a pseudo-racemate) of two or more substrate molecule types, such as mixtures of pseudo-stereoisomers (e.g., pseudo-enantiomers, pseudo-diastereomers, or the like). In other embodiments, the substrate molecule types include pseudo-meso compounds. Substrate molecule types typically include one or more cyclic or acyclic organic compounds. In certain preferred embodiments, the substrate molecule types are esters.

In preferred embodiments, the enzyme (e.g., an artificially evolved enzyme) is a member of an expression library and the method includes screening (e.g., sequentially, in parallel, or the like) two or more members of the expression library for enzyme stereoselectivity. Typically, one or more of the two or more products include the at least one of the one or more leaving groups (e.g., acyl, alcohol, or other moieties). For example, two of the two or more products optionally include pseudo-enantiomers, or at least two of the two or more products optionally include pseudo-diastereomers. In certain embodiments, the products are quantified by liquid chromatography mass spectrometry, by gas chromatography mass spectrometry, by capillary electrophoresis mass spectrometry, or the like. The methods typically further include comparing amounts of quantified products with one another or with a control. Optionally, the methods further include comparing a ratio of amounts of quantified products with a control. In addition, the methods provide a measure of initial reaction kinetics when the products are detected when conversion of substrate(s) to product(s) is about 10% or less.

### BRIEF DESCRIPTION OF THE DRAWING

FIG. 1 schematically shows the hydrolysis of pseudo-meso(1S,3R)-1-deuterobutanoyl-3-butanoylcyclopentane to form a mixture of pseudo-enantiomers.

FIG. 2 schematically depicts the hydrolysis of a mixture that includes neryl butyrate and geranyl deuterobutyrate to yield products, butyrate and deuterobutyrate (in boxes), that can be detected mass spectrometrically.

FIG. 3 provides data graphs showing the quantification of different ratios of butyrate (top histogram) and deuterobutyrate (bottom histogram) simultaneously by mass spectrometry.

### DETAILED DISCUSSION OF THE INVENTION

The present invention involves the use of isotopically labeled substrate molecules that, upon enzymatic conversion, release an isotopically labeled product (i.e., an isotopically labeled substrate leaving group) that can be detected by mass spectrometry. In particular, the methods of the invention include screening expression libraries for enzyme stereoselectivity by contacting library members with substrate mixtures, such as mixtures of pseudo-stereoisomers, such as pseudo-racemates, or with pseudo-meso compounds. For example, in certain embodiments, the substrate mixture includes pseudo-stereoisomers of a substrate molecule, such as an ester or other organic molecule that has a leaving group (e.g., an acyl, an alcohol, or other moiety) with three or more carbon atoms, in which a leaving group of at least one pseudo-stereoisomer is isotopically labeled. In accordance with the present invention, upon enzymatic conversion of the substrate, the isotopically labeled leaving group becomes the product that is detected by mass spectrometry. In particular, the present invention provides a sensitive method for measuring enzyme selectivity at conversions of about 10% or less, more particularly at conversions of about 5% or less, and sometimes at conversions of about 3% or less. The methods are even suitable for measuring enzyme selectivity at conversions of about 1% or less.

In overview, the following discussion provides details relating to substrate molecule selection and preparation (e.g., isotopically labeling, etc.). It also describes many different techniques for generating libraries of artificially evolved enzymes for screening. These techniques include, e.g., the recombination (e.g., recursive sequence recombination, whole genome recombination, or the like) and/or the mutation (e.g., site directed mutagenesis, cassette mutagenesis, random mutagenesis, recursive ensemble mutagenesis, in vivo mutagenesis, or the like) of one or more nucleic acids that encode the enzymes (e.g., hydrolases, such as lipases, esterases, or the like) to be screened. The discussion additionally relates to various system components, including those for handling, e.g., cell cultures, substrate molecules and other reagents, or the like. Furthermore, details pertaining to mass spectrometric detection and quantification of reaction products are also provided.

I. Definitions

Unless defined otherwise, all technical and scientific terms used herein have the meaning commonly understood by a person skilled in the art to which this invention belongs. The following references provide one of skill with a general definition of many of the terms used in this invention: Müller et al. (1994) “Glossary of terms used in physical organic chemistry,” Pure Appl. Chem. 66:1077-1184 and Achmatowicz et al. (1996) “Basic terminology of stereochemistry,” Pure Appl. Chem. 68:2193-2222.

The phrase “enzyme stereoselectivity” refers to the preferential formation of one stereoisomer or pseudo-stereoisomer over another or others in a chemical reaction catalyzed by an enzyme. When the stereoisomers are enantiomers, the phenomenon is referred to as “enzyme enantioselectivity” and is quantitatively expressed by the enantiomeric excess; when the stereoisomers are diastereoisomers, it is called “enzyme diastereoselectivity” and is quantitatively expressed by the diastereoisomeric excess. “Enantiomeric excess” refers to the absolute difference between the mole or weight fractions of major (F₍₊₎) and minor (F₍₋₎) enantiomers (i.e., |F₍₊₎−F₍₋₎|), where F₍₊₎+F₍₋₎=1. The percent enantiomer excess is 100× |F₍₊₎−F₍₋₎|. “Diastereoisomeric excess” refers to the absolute difference between the mole or weight fractions of major (D₍₊₎) and minor (D₍₋₎) diastereomers (i.e.,|D₍₊₎−D₍₋₎|), where the mole or weight fractions of two diastereomers in a mixture or the fractional yields of two diastereomers formed in a reaction are D(+) and D(−) (i.e., D₍₊₎+D₍₋₎=1). The percent diastereoisomeric excess is 100×|D₍₊₎−D₍₋₎|.

“Stereoisomers” are isomers that possess an identical constitution, but which differ in the arrangement of their atoms in space. “Pseudo-stereoisomers” are stereoisomers that differ in isotopic labeling. For example, neryl butyrate and geranyl deuterobutyrate are pseudo-stereoisomers.

“Constitution” refers to the description of the identity and connectivity (and corresponding bond multiplicities) of the atoms in a molecular entity (omitting any distinction arising from their spatial arrangement).

The term “percent conversion” refers to the enzymatic conversion of substrate and is computed according to the following: %conversion=100×(Sᵢₙᵢₜᵢₐₗ−Sₜ)/Sᵢₙᵢₜᵢₐₗ, where Sᵢₙᵢₜᵢₐₗ is the initial concentration of total substrate. The quantity (Sᵢₙᵢₜᵢₐₗ−Sₜ) is equal to the total amount of product generated at time t. and Sₜ is the substrate mixture concentration at a timepoint in the reaction, t, and is equal to the initial concentration of substrate mixture minus total converted product at time t.

“Enantiomers” are stereoisomers that are nonsuperimposable mirror images of one another. “Pseudo-enantiomers” are enantiomers that differ in isotopic labeling.

“Diastereomers” are stereoisomers that are not enantiomers. “Pseudo-diastereomers” are diastereomers that differ in isotopic labeling.

A “meso compound” is a compound that includes asymmetric carbons, but which is achiral due to a plane of symmetry. “Pseudo-meso compounds” are meso compounds that differ in isotopic labeling.

A “mixture” refers to a combination of two or more different molecules in varying proportions in which the different molecules retain their own properties.

A “pseudo-racemate” refers to an equimolar mixture of a pair of pseudo-enantiomers.

An “organic” chemical compound or substituent group is one that includes at least one carbon atom, but which also typically includes additional substituent or functional groups, such as amino, alkoxy, cyano, hydroxy, carboxy, halo, acyl, alkyl, cycloalkyl, hetaryl, aryl, allylic, vinylic, arylene, benzylic, or derivatives thereof and/or other groups or derivatives thereof. Organic compounds or substituent groups are cyclic or acyclic. Exemplary organic compounds or substituent groups include esters, ketones, alcohols, epoxides, polyols, ethers, phenols, aldehydes, quinones, carboxylic acids, derivatives thereof, or the like.

“Esters” are a class of organic compounds that include the general formula RCOOR′, where R and R′ are any alkyl or aryl groups. Esters are an example of one class of compounds that are utilized as substrate molecules according to the methods described herein.

“Alcohol” refers to an organic molecule or group that includes at least one hydroxy group.

“Polyol” refers to an organic molecule or group that includes two or more hydroxy groups.

“Epoxide” refers to an organic molecule or group that includes at least one oxygen atom in a three-membered ring (i.e., a cyclic ether).

“Acyl moieties” refer to organic groups that include the general formula RCO—, where R is any alkyl, aryl, or alkylaryl group.

“Alcohol moieties” refer to organic groups that include at least one hydroxy group (—OH).

A “leaving group” refers to an atom or moiety (charged or uncharged) that becomes displaced or cleaved from a substrate molecule in a chemical reaction. For example, a leaving group from the hydrolysis of an ester can include, e.g., an acyl, an alcohol, and/or other moiety.

A “moiety” refers to one of the portions into which something, such as a substrate molecule is divided (e.g., a functional group, substituent group, or the like). For example, esters include acyl, alcohol, and/or other moieties.

Reaction “kinetics” refers to the rate and mechanism by which one chemical species is converted into another. See, e.g., Steinfeld, Chemical Kinetics and Dynamics, 2^{nd} Ed., Prentice-Hall, Inc. New Jersey (1999).

A “detection limit” is the minimum concentration or mass of analyte (e.g., a reaction product) that can be detected at a known confidence level.

The “sensitivity” of an instrument or a method is a measure of its ability to discriminate between small differences in analyte concentration.

A “condensation reaction” refers to a reaction in which two or more atoms or molecules combine into a larger molecule with or without the loss of a small molecule.

A “hydrolysis reaction” refers to a reaction with water involving the rupture of one or more bonds in the reacting solute (e.g., a substrate molecule).

A “hydrolase” refers to any member of the class of enzymes that catalyze the hydrolysis of chemical bonds. Exemplary hydrolases include lipases, esterases, phosphorylases, glycosidases, nucleases, proteases, and the like. See also, the ENZYME nomenclature database (www.expasy.ch/enzyme/) at the ExPASy proteomics server of the Swiss Institute of Bioinformatics, and Bairoch (2000) “The ENZYME database in 2000” Nucleic Acids Res. 28:304-305.

An “enzyme” refers to a protein that acts as a catalyst to reduce the activation energy of a chemical reaction involving other compounds or “substrates.”

An “artificially evolved enzyme,” refers to a protein- or nucleic acid-based catalyst or enzyme (e.g., a hydrolase or the like), created using one or more diversity generating techniques. For example, artificially evolved enzymes employed in the practice of the present invention are optionally produced by recombining (e.g., via recursive recombination, whole genome recombination, synthetic recombination, in silico recombination, or the like) two or more nucleic acids encoding one or more parental enzymes, or by mutating one or more nucleic acids that encode enzymes, e.g., using site directed mutagenesis, cassette mutagenesis, random mutagenesis, recursive ensemble mutagenesis, in vivo mutagenesis, or the like. A nucleic acid encoding a parental enzyme includes a polynucleotide or gene that, through the mechanisms of transcription and translation, produces an amino acid sequence corresponding to a parental enzyme, e.g., an unevolved or naturally-occurring hydrolase. The term, “artificially evolved enzymes” also embraces chimeric enzymes that include identifiable component sequences (e.g., functional domains, etc.) derived from two or more parents. Artificially evolved enzymes employed in the practice of the present invention are typically evolved to yield products stereoselectively. Diversity generating methodologies that are optionally used to produce the artificially evolved enzymes of the present invention are discussed in greater detail below.

A “library” refers to a collection of at least two different molecules, such as nucleic acid sequences or expression products (e.g., enzymes) derived therefrom. A library generally includes large numbers of different molecules. For example, a library typically includes at least about 100 different types of molecules, more typically at least about 1000 different types of molecules, and often at least about 10000 or more different types of molecules. A “library” or “expression library” optionally includes naturally occurring enzymes and/or artificially evolved enzymes.

A “mass spectrometer” is an analytical instrument that can be used to determine the molecular weights of various substances, such as products of an enzyme catalyzed reaction. Typically, a mass spectrometer comprises four parts: a sample inlet, an ionization source, a mass analyzer, and a detector. A sample is optionally introduced via various types of inlets, e.g., solid probe, gas chromatography column (GC), or liquid chromatography column (LC), in gas, liquid, or solid phase. The sample is then typically ionized in the ionization source to form one or more ions. The resulting ions are introduced into and manipulated by the mass analyzer. Surviving ions are detected based on mass to charge ratios. In one embodiment, the mass spectrometer bombards the substance under investigation with an electron beam and quantitatively records the result as a spectrum of positive and negative ion fragments. Separation of the ion fragments is on the basis of mass to charge ratio of the ions. If all the ions are singly charged, this separation is essentially based on mass. A quadrupole mass spectrometer uses four electric poles for the mass analyzer. These techniques are described generally in many basic texts, e.g., Dawson, Quadrupole Mass Spectrometry and its Applications, Springer Verlag, (1995). In an electrospray mass spectrometry system, ionization is produced by an electric field that is used to generate charged droplets and subsequent analyte ions by ion evaporation. See, Cole “Electrospray Ionization Mass Spectrometry” John Wiley and Sons, Inc. (1997).

A “cell growth plate” refers to a plate on which cell colonies can be grown in an appropriate media. Exemplar plates include 1536, 384, or 96-well microtiter plates. For example cell colonies containing gene libraries are picked directly from transformation plates into 1536, 384, or 96-well microtiter plates with appropriate growth media using, e.g., a Q-bot from Genetix (www.genetix.co.uk).

An “automatic sampler” is a robotic handler that transports samples from one location to another. An automatic sampler is used for example, to transport samples from a cell growth plate and inject them into a mass spectrometer for analysis. Examples of automatic samplers include microtiter autosamplers available from OmniLab Biosystems AG, Gilson, Inc., and CTC Analytics. Automatic samplers optionally include robotic handlers that are used to pick colonies, such as a Q-bot available from Genetix, and/or add or remove reagents to or from the cell growth plate.

The term “substrate molecule type” refers to a species of stereoisomer. The plural form, “substrate molecule types” refers to different species of stereoisomers.

“Derivative” refers to a chemical substance related structurally to another substance, or a chemical substance that can be made from another substance (i.e., the substance it is derived from), e.g., through chemical or enzymatic modification.

II. The Methods and Systems of the Invention

The present invention generally provides a method of screening for enzyme stereoselectivity, comprising:

providing a plurality of substrate molecules, wherein the plurality comprises two or more substrate molecule types, wherein at least one of the substrate molecule types has one or more leaving groups, wherein at least one of the leaving groups is isotopically labeled;

contacting at least one enzyme with the plurality of substrate molecules, wherein the enzyme converts one or more of the substrate molecules to two or more products, wherein at least one of the products comprises the isotopic label; and

quantifying the two or more products mass spectrometrically, thereby screening for enzyme stereoselectivity.

Typically the plurality of substrate molecules is made up of substrate molecule types that are either different pseudo-stereoisomers, different pseudo-meso compounds, or different pseudo-diasteromers. Usually the plurality of substrate molecules is a racemic mixture.

The present invention further provides a method of screening for enzyme stereoselectivity, comprising

providing a pseudo-meso substrate molecule comprising at least one isotopically labeled leaving group;

contacting at least one enzyme with the pseudo-meso substrate molecule, wherein the enzyme converts the pseudo-meso substrate molecule to two or more products,

quantifying the two or more products mass spectrometrically, wherein at least one of the quantified products comprises the isotopically labeled leaving group, thereby screening for enzyme stereoselectivity.

The present invention is particularly suitable for screening an enzyme library for enzyme selectivity. Enzyme libraries of, for example, naturally occurring or artificially evolved enzymes, are typically generated by expression on cell growth plates as described herein. The cell growth plate optionally contains the plurality of substrate molecules, and the cell growth plate is optionally maintained under conditions that facilitate the conversion of substrate to product by members of the enzyme library. An autosampler can be used to transport product samples from the cell growth plate to the mass spectrometer for injection and analysis. These methods are described in more detail herein below.

The invention methods are particularly suitable for quantifying the enzymatically converted products at low percent conversion of substrate(s). The invention methods are suitable for determining enzyme stereoselectivity under initial kinetic conditions, where conversion is typically about 10% or less. Methods of the present invention can be employed to determine quantities of converted product even when the conversion of substrate to product is only about 5% or less, and sometimes when the conversion of substrate to product is about 3% or less, or even about 1% or less.

Once the amount of converted product is determined, enzyme stereoselectivity can be readily assessed by, for example, computing enantiomeric excess values or ratios of amounts of each product quantified (e.g., quantity or concentration of unlabeled product divided by quantity or concentration of isotopically labeled product, or vice-versa). A comparative screen can also be conducted by utilizing a control/reference enzyme, and comparing the selectivity of the enzyme of interest to that of the control/reference enzyme.

A. Substrate Molecules

Essentially a plurality of substrate molecules that is any set of pseudo-stereoisomers, pseudo-meso compounds, or pseudo-diasteromers is contacted with an enzyme (e.g., a naturally occurring enzyme, an artificially evolved enzyme, or the like) to screen for enzyme stereoselectivity according to the methods described herein. As a consequence, no attempt is made herein to describe all suitable substrate molecules. Appropriate substrate molecules will be readily apparent to one of skill in the art, e.g., in view of desired products, which typically include compounds of pharmaceutical, industrial, agricultural, or other significance. In certain embodiments, substrate molecules are members of combinatorial chemical libraries. Many substrate molecules optionally utilized with the methods of the present invention are described in the references cited herein.

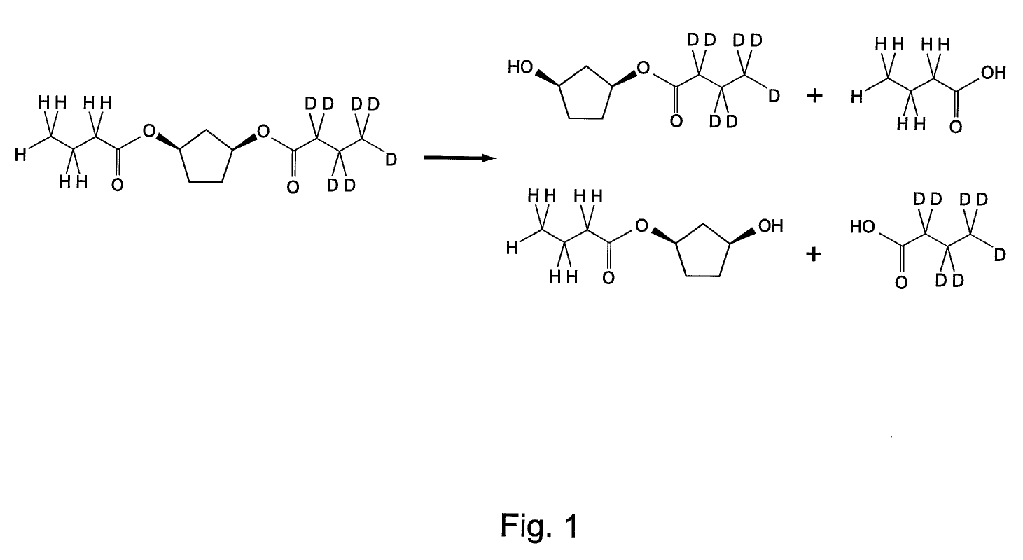

In certain preferred embodiments, mixtures of substrate molecules include pseudo-stereoisomer substrate molecules, such as esters having leaving groups (e.g., acyl, alcohol, and/or other moieties) that include three or more carbon atoms, or in certain other preferred embodiments, four or more carbon atoms. For example, larger acyl cleavage products (e.g., isotopically labeled products) typically lead to increased sensitivity upon detection relative to products having fewer than three carbon atoms such as acetyl moieties. Mixtures of pseudo-stereoisomers utilized in the screening methods of the invention typically include pseudo-enantiomers, pseudo-diastereomers, or the like. An example screen that employs a mixture of pseudo-diastereomers, namely, neryl butyrate and geranyl deuterobutyrate is provided below. In certain embodiments, mixtures of, e.g., more than two pseudo-diastereomer substrate molecules are optionally included. In some preferred embodiments, the methods include providing pseudo-racemates of pseudo-enantiomers for analysis. In other preferred embodiments, the methods include screening for enzyme stereoselectivity by contacting enzymes (e.g., from an expression library) with pseudo-meso compounds that also have leaving groups (e.g., acyl, alcohol, and/or other moieties), which include three, four, or more carbon atoms. For example, FIG. 1 schematically shows the hydrolysis of pseudo-meso-(1S,3R)-1-deuterobutanoyl-3butanoylcyclopentane to form a mixture of pseudo-enantiomers, namely, (1R,3S)-1-butanoylcyclopentan-3-ol and (1S, 3R)-1-deuterobutanoylcyclopentan-3-ol.

Individual substrate molecules within a given mixture are typically differentiated from one another by the inclusion of one or more distinguishing isotopic labels (i.e., to form pseudo-stereoisomers, pseudo-meso compounds, etc.). To illustrate, an enzyme is optionally screened for stereoselectivity towards pseudo-enantiomers of, e.g., propyl-3-hydroxybutanoate, butyl3-hydroxybutanoate, and the like. In the preparation of these pseudo-enantiomers, for example, the acyl or alcohol moiety of one pseudo-enantiomer is optionally synthesized with, e.g., one or more deuterium substitutions, whereas the other pseudo-enantiomer is synthesized without such isotopic labels. Alternatively, both pseudo-enantiomers include isotopic labels, e.g., different numbers of the same isotopic label and/or different isotopic labels. The leaving groups of meso compounds utilized in the screens of the invention are optionally similarly labeled. Suitable isotopic labels are generally known in the art and include, e.g., ²H, ³H, ⁷Li, ¹³C, ¹⁴C, ¹¹B, ¹⁹F, ³¹P, ³²P, ¹⁵N, ¹⁷O, ¹⁸O, or the like.

Substrate molecules, including isotopically labeled molecules, are optionally synthesized according to known methods or purchased from commercial suppliers. For example, various synthetic techniques for forming esters or other substrate molecules and isotopically labeling compounds are generally known and described in, e.g., March, Advanced Organic Chemistry: Reactions, Mechanisms, and Structure, 4^{th} Ed., John Wiley & Sons, Inc., New York (1992), Carey and Sundberg, Advanced Organic Chemistry Part A: Structure and Mechanism, 4th Ed., Plenum Press, New York (2000), and in the references provided therein. Commercial suppliers of chemical substrates, including isotopically labeled substrate molecules are also known and include, e.g., Sigma-Aldrich, Inc. (St Louis, Mo.)(www.sigma-aldrich.com), Martek Biosciences Corporation (Columbia, Md.)(www.martekbio.com), Cambridge Isotope Laboratories, Inc. (Andover, Mass.)(www.isotope.com), Medical Isotopes, Inc. (Pelham, N.H.) (www.medicalisotopes.com), Isotec Inc. (Miamisburg, Ohio) (www.isotec.com), Silantes GmbH (München, Germany)(www.silantes.com), C/D/N ISOTOPES Inc. (Quebec, Canada) (www.cdniso.com), or the like.

B. Cell Growth Plates

The cell growth plates of the invention are optionally 1536, 384, or 96-well microtiter plates, or the like. For example, cell colonies containing gene libraries are picked directly from transformation plates into 1536, 384, or 96-well microtiter plates containing appropriate growth media using, for example, a Q-bot from Genetix. The maximum speed of the Q-bot is about 4000 colonies per hour.

The microtiter plates are typically incubated in a plate shaker for cell growth, e.g., typically for 1 day to about 2 weeks depending on the organism. Media and cell growth conditions are appropriate to the particular cells that are incubated.

The cell growth plate is also typically utilized for product generation when, for example, enzyme reactions are being screened, e.g., according to the methods of the present invention. Products of reactions between enzymes and substrate molecules are of interest when evolving new functional enzymes. These products (and optionally, the reactants) is/are typically analyzed in a high-throughput method so that many members of the enzyme library can be analyzed in a short period of time. To allow high-throughput measurement of the products, they are optionally generated as part of the automated system of the invention. Therefore, any product generation steps that must be undertaken in the assay are optionally performed on the cell growth plate. After generation of products, the samples, which contain the products, are optionally purified for injection into a mass spectrometer for analysis.

C. Autosampler

An autosampler is typically included in the systems of the invention to transport samples between the cell growth plate, where cells are grown and reactants and/or products of interest are generated and purified, to the mass spectrometer for injection and analysis. Autosamplers can be purchased from standard laboratory equipment suppliers such as OmniLab Biosystems AG, Gilson, Inc., and CTC Analytics. Such samplers typically function at rates of about 10 seconds/sample to about 1 min/sample.

In addition, robotic sample handlers are optionally used to pick cell colonies into the cell growth plate and to additionally add reagents thereto. For the generation of common arrangements involving fluid transfer to or from microtiter plates, a fluid handling station is used. Such robotic handlers include but are not limited to those produced by Beckman Instruments and Genetix (e.g., the Q-bot). In addition, several “off the shelf” fluid handling stations for performing such transfers are commercially available, including e.g., the Zymate systems from Zymark Corporation (Zymark Center, Hopkinton, Mass.; www.zymark.com/) and other stations which utilize automatic pipettors, e.g., in conjunction with the robotics for plate movement, e.g., the ORCA® robot, which is used in a variety of laboratory systems available, e.g., from Beckman Coulter, Inc. (Fullerton, Calif.).

Robotic sample handlers are also optionally used to remove enzymes from a cell growth plate as described above. For example, a robotic handler is optionally used to lift a set of pins from a reaction well or to position a magnet to lift a set of magnetic beads from a cell growth plate, e.g., beads comprising a tagged enzyme.

D. Enzyme Selectivity Screening and Mass Spectrometric Analysis

Screening methods of the present invention include the steps of contacting enzymes, such as artificially evolved enzymes from one or more libraries, with mixtures of pseudo-stereoisomers (e.g., pseudo-enantiomers, pseudo-diastereomers, or the like) or with pseudo-meso compounds, and detecting and quantifying by mass spectrometry labeled and unlabeled products that are generated, to identify enzymes that selectively convert pseudo-stereoisomers or pseudo-meso-compounds. Techniques for performing enzyme catalyzed reactions and for detecting reaction products are generally known in the art. A discussion of methods of generating nucleic acids that encode artificially evolved enzyme libraries is provided below. In preferred embodiments, the mixture includes ester pseudo-stereoisomers and the reaction involves the hydrolysis (e.g., acyl cleavage or the like) of one or more of the isomers catalyzed by a hydrolase (e.g., a lipase). Optionally, pseudo-meso ester compounds are screened according to the methods of the invention. In other embodiments, enzymes that catalyze condensation reactions are optionally screened according to the methods described herein. One advantage of these screening methods is that specific products can be detected and quantitated, even from a complex mixture of products and substrates, thus providing a direct measurement of enzyme selectivity, e.g., enantioselectivity, diastereoselectivity, or the like.

Mass spectrometry is an analytical technique that is typically used to provide information about, e.g., the isotopic ratios of atoms in samples, the structures of various molecules, and the qualitative and quantitative composition of complex mixtures. Common mass spectrometer systems include a system inlet, an ion source, a mass analyzer, and a detector that are under vacuum. The detector is typically operably connected to a signal processor and a computer. Desorption ion sources employed in the practice of the present invention optionally include field desorption (FD), electrospray ionization (ESI), chemical ionization, matrix-assisted laser desorption/ionization (MALDI), plasma desorption (PD), fast atom bombardment (FAB), secondary ion mass spectrometry (SIMS), thermospray ionization (TS), or the like. A variety of mass spectrometer instruments are commercially available. For example, Micromass (U.K.) produces a variety of suitable instruments such as the Quattro LC (a compact triple stage quadrupole system optimized, e.g., for API LC-MS-MS) which utilizes a dual stage orthogonal “Z” spray sampling technique. Other suitable triple stage quadrupole mass spectrometers (e.g., the “TSQ” spectrometer) are produced by the Finnigan Corporation.

Mass spectrometry (MS) is a generic method that allows detection of a large variety of different small molecule metabolites. Ionspray and electrospray mass spectrometry have been used in many different fields for the analysis of organic compounds. It is however, usually coupled to a separation technique, such as liquid chromatography, gas chromatography, or capillary zone electrophoresis, which is performed in-line with the mass spectrometry analysis. Thus, subsequent to conversion of some substrate to product(s), one or more of these separation techniques may be conducted on the product samples prior to analysis by mass spectrometry. Methods of performing high throughput mass spectrometry screening that are adaptable for use with the methods of the present invention are described in, e.g., International Patent Application PCT/US00/03686 entitled “HIGH THROUGHPUT MASS SPECTROMETRY,” by Raillard et al., which was filed Feb. 11, 2000. See also, Reetz et al. (1999) “A method for high-throughput screening of enantioselective catalysts,” Agnew. Chem. Int. Ed. 38(12):1758-1761 and Bakhtiar and Tse (2000) “Biological mass spectrometry: a primer,” Mutagenesis 15(5):425-430. General sources of information about mass spectrometry include, e.g., Kirk-Othmer Encvclopedia of Chemical Technology, Vol. 15, 4th Ed., pages 1071-1094, and all references therein. See also, Siuzdak, Mass Spectrometry for Biotechnology, Academic Press, San Diego (1996), Cole (Ed.), Electrospray Ionization Mass Spectrometry: Fundamentals, Instrumentation, and Applications, Wiley and Sons, Inc., New York (1997), Johnstone et al., Mass Spectrometry for Chemists and Biochemists, Cambridge University Press, Cambridge (1996), Hoffman et al., Mass Spectrometry: Principles and Applications, Wiley and Sons, Inc. (1996), Dawson (Ed.), Quadrupole Mass Spectrometry and its Applications, Springer Verlag, (1995), Karjalainen et al. (Eds.), Advances in Mass Spectrometry, Elsevier Science, (1998), and Skoog et al., Principles of Instrumental Analysis (5 ^{th} Ed.) Hardcourt Brace & Company, Orlando (1998).

Electrospray methods are optionally used instead of gas chromatography procedures because no prior derivatization is required to inject the sample. Flow injection analysis (FIA) methods with ionspray-ionization and tandem mass spectrometry further the ability of the present invention to perform high-throughput mass spectrometry analysis. The ionspray method allows the samples to be injected without prior derivatization and the tandem mass spectrometry (MS/MS) allows extremely high efficiency in the analysis. Therefore, no column separation is needed.

Electrospray ionization is a very mild ionization method that allows detection of molecules that are polar and large which are typically difficult to detect in GC-MS without prior derivatization. Modem electrospray mass spectrometers detect samples in femtomole quantities. Since a couple of microliters are injected, samples are optionally injected in nanomolar concentrations, attomolar concentrations or lower. Quantitation is very reproducible with standard errors ranging from 2%-5%.

Tandem mass spectrometry uses the fragmentation of precursor ions to fragment ions within a triple quadrupole MS. The separation of compounds with different molecular weights occurs in the first quadrupole by the selection of a precursor ion. The identification is performed by the isolation of a fragment ion after collision induced dissociation of the precursor ion in the second quadrupole. Reviews of this technique can be found in Kenneth, L. et al. (1988) “Techniques and Applications of Tandem Mass Spectrometry” VCH publishers, Inc.

Triple quadrupole mass spectrometers allow MS/MS analysis of samples. For example, a triple quadrupole mass spectrometer with electrospray and atmospheric pressure chemical ionization sources, such as a Finnigan TSQ 7000, is optionally used. The machine is optionally set to allow one particular parent ion through the first quadrupole which undergoes fragmentation reactions with an inert gas. The most prominent daughter ion can then be singled out in the third quadrupole. This method creates two checkpoints for analyte identification. The particle must have the correct molecular mass to charge ratio of both parent and daughter ion. Tandem mass spectrometry thus leads to higher specificity and often also to higher signal to noise ratios. It also introduces further separation by distinguishing analyte from impurities with same mass to charge ratio.

Other techniques optionally used in the present invention include, but are not limited to, neutral loss and parent ion scanning. Neutral loss is a method of mass spectrometry scanning in which all compounds that lose a neutral molecular fragment, i.e., a specific neutral fragment, during collision induced dissociation (CID) are detected. Parent ion mode detects all compounds that produce a common daughter ion fragment during CID. These techniques are optionally used, e.g., to quantitate the amount of product and starting material simultaneously. For systems in which the expected product is not known, e.g., a standard is not available, the neutral loss and/or parent ion method allows backtracking or deconvolution based on fragmentation patterns to determine the structure and/or identity of the starting material. For example, the parent mass is determined based on the various fragments produced. This is especially useful for detecting novel enzyme activity when the product of the enzyme reaction is not known, but is predictable.

In neutral loss methods, components of interest are allowed to pass the first quadrupole, e.g., in a triple quadrupole spectrometer, one at a time by scanning the first quadrupole in a certain mass range. The components, e.g., ions, are fragmented in the second mass filter by CID. If a specific neutral fragment is lost from a parent ion during the CID process, a daughter ion is formed, which daughter ion has a mass equal to the mass of the parent ion minus the mass of the neutral molecule. The daughter ion will pass the third filter and be detected. In this way, any ion or components losing a neutral fragment, e.g., a constant neutral fragment (N₀) during the CID process in the second quadrupole is optionally detected by scanning the first and third quadrupoles simultaneously with a mass offset equal to the mass N₀.

In the parent ion method, ions or components of interest are allowed to pass the first quadrupole one at a time. These ions are fragmented in a second mass filter by CID. The third quadrupole is then set to allow only specific ions to pass. Thus, all components, e.g., products or reactants, producing a specific fragment ion as set in the second quadrupole are detected by scanning the first quadrupole mass filters in the range of interest while setting the third quadrupole mass filter on that specific ion.

The speed of the analysis is limited only by the motoric movements of the autosampler used to inject the samples, such as a CTC Analytics and Gilson, Inc. (www.gilson.com). The speed for example, is optionally set at 30 seconds without wash and 40 seconds with wash of the injection needle. Such a sampling rate allows 2880 samples per day to be analyzed by MS if automated overnight runs are used. Thus, an entire 96-well microtiter plate of samples is run in less than an hour. Preferably, the speed of the autosampler is set at about 15 seconds per sample, allowing about 5000 samples to be screened in one day or about 200 per hour. Autosampler companies are currently working to increase the throughput to one plate in 10 minutes including the washing, which would then allow for about 8500 MS samples to be run in a day.

The rate of screening is optionally increased beyond that of the autosampler by using pooling strategies, e.g., with the neutral loss, parent ion screening methods described above. A plurality of samples, e.g., similar or related samples, are optionally pooled or mixed together and injected into the mass spectrometer as one sample. The data is then deconvoluted to provide identification or analysis for each of the pooled samples. For example, five different substrates are reacted with an enzyme and the results pooled. The five different substrates may produce five related or similar compounds as products. The products are pooled and analyzed. Neutral loss analysis is then optionally performed on the pooled samples. For example, a specified neutral fragment is removed from all the samples, e.g., in the second quadrupole, and then the data is deconvoluted to determine the parent ion as detected in the first quadrupole to provide results for each of the individual samples.

E. Computer Interface

Control of the elements of the system and/or the analysis of detected system information are coupled to an appropriately programmed processor or computer, or computer readable medium which functions to instruct the operation of these instrument elements in accordance with preprogrammed or user input instructions, receive data and information from these instruments, and interpret, manipulate and report this information to the user. As such, the computer is typically appropriately coupled to any library storage elements, injection elements, and/or the MS, and/or to any analog to digital or digital to analog converter element as desired.

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing movement of library elements, control of the MS and the like. The computer then receives the data from one or more signal sensor/detectors included within the MS system, and interprets the data, either providing it in a user interpretable format, or uses that data to initiate further instructions, in accordance with the programming, e.g., such as in monitoring and control of injection rates, library selection, temperatures, applied fields, or the like.

In the present invention, the computer typically includes software for the monitoring of materials in the MS. Additionally the software is optionally used to control injection or withdrawal of material into or from the MS. The injection or withdrawal is used to select and quantify library members, or products of reactions catalyzed thereby, in the system.

In general, one or more instruction sets are present in the computer, or on a computer-readable medium such as a computer hard-drive or CD-ROM that includes instruction sets for MS operation and signal detection/deconvolution. Instruction sets exist in computer memory or on a computer-readable medium such as a computer hard-drive or CD-ROM and are provided by the present invention and accessed by the system for the operation of the instruction sets.

Typically, a computer commonly used to transform signals from the detection device into reaction rates will be a PC-compatible computer (e.g., having a central processing unit (CPU) compatible with x86 CPUs (e.g., a Pentium I, II or III class machine), and running an operating system such as LINUX, DOSTM, OS/2 Warp™, WINDOWS/NT™, WNDOWS/NT™ workstation, or WINDOWS 98™), or a Macintosh™ (running MacOS™), or a UNIX workstation (e.g., a SUN™ workstation running a version of the Solaris™ operating system, a PowerPC™ workstation or a mainframe computer), all of which are commercially common, and known to one of skill in the art. Data analysis software on the computer is then employed to deconvolute signal information. Software for these purposes is available, or can easily be constructed by one of skill using a standard programming language such as Visual Basic, Fortran, Basic, Java, or the like.

One of skill will readily recognize that any, or all, of these components can be optionally manufactured in separable modular units, and assembled to form an apparatus or system of the invention. Computers, MS detectors, library manipulation robots, and the like are optionally manufactured in a single unit, but more commonly are constructed as separate modules which are assembled to form an apparatus or system for analyzing a library of components. Further, a computer does not have to be physically associated with the rest of the apparatus to be “operably linked” to the apparatus. A computer is operably linked when data is delivered from other components of the apparatus to the computer. One of skill will recognize that operable linkage can easily be achieved using either conductive cable coupled directly to the computer (e.g., USB, parallel, serial, ethernet, or phone line cables), or using data recorders which store data to computer readable media (typically magnetic or optical storage media such as computer disks and diskettes, CDs, magnetic tapes, but also optionally including physical media such as punch cards, vinyl media or the like) which is then accessed by the computer.

F. Artificially Evolved Enzyme Libraries

The methods of the present invention typically include screening libraries of naturally occurring and/or artificially evolved enzymes, i.e., using the mass spectrometry-based methods and systems of the invention. A variety of diversity generating protocols for artificially evolving enzymes (e.g., nucleic acids encoding artificially evolving enzymes) are available and described in the art. The procedures can be used separately, and/or in combination to produce one or more variants of a nucleic acid or set of nucleic acids, as well variants of encoded enzymes that are optionally screened according to the methods described herein. Individually and collectively, these procedures provide robust, widely applicable ways of generating diversified nucleic acids and sets of nucleic acids (including, e.g., nucleic acid libraries) useful, e.g., for the engineering or rapid evolution of nucleic acids, proteins, pathways, cells and/or organisms with new and/or improved characteristics, such as the ability to stereoselectively catalyze a desired reaction.

While distinctions and classifications are made in the course of the ensuing discussion for clarity, it will be appreciated that the techniques are often not mutually exclusive. Indeed, the various methods can be used singly or in combination, in parallel or in series, to access diverse sequence variants.

The result of any of the artificial evolution procedures described herein can be the generation of one or more nucleic acids, which can be selected or screened for nucleic acids that encode proteins with or which confer desirable properties. Following diversification by one or more of the methods herein, or otherwise available to one of skill, any nucleic acids that are produced can be selected for a desired activity or property, e.g., an ability to stereoselectively catalyze a given reaction. This can include identifying any activity that can be detected, for example, in an automated or automatable format, by any of the assays described herein. Optionally, a variety of related (or even unrelated) properties can be evaluated, in serial or in parallel, at the discretion of the practitioner.

The following publications describe a variety of recursive recombination procedures and/or methods which can be incorporated into such procedures: Stemmer, et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding”

Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer” Journal of Molecular Biology 255:373-386; Stemmer (1996) “Sexual PCR and Assembly PCR” In: The Encyclopedia of Molecular Biology. VCH Publishers, New York. pp. 447-457; Crameri and Stemmer (1995) “Combinatorial multiple cassette mutagenesis creates all the permutations of mutant and wildtype cassettes” BioTechniques 18:194-195; Stemmer et al. (1995) “Single-step assembly of a gene and entire plasmid form large numbers of oligodeoxyribonucleotides” Gene, 164:49-53; Stemmer (1995) “The Evolution of Molecular Computation” Science 270:1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751.

Mutational methods of generating diversity include, for example, site-directed mutagenesis (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein and Shortle (1985) “Strategies and applications of in vitro mutagenesis” Science 229:1193-1201; Carter (1986) “Site-directed mutagenesis” Biochem. J. 237:17; and Kunkel (1987) “The efficiency of oligonucleotide directed mutagenesis” in Nucleic Acids & Molecular Biology (Eckstein, F. and Lilley, D. M. J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100:468-500 (1983); Methods in Enzymol. 154:329-350 (1987); Zoller and Smith (1982) “Oligonucleotide-directed mutagenesis using M13-derived vectors: an efficient and general procedure for the production of point mutations in any DNA fragment” Nucleic Acids Res. 10:6487-6500; Zoller and Smith (1983) “Oligonucleotide-directed mutagenesis of DNA fragments cloned into M13 vectors” Methods in Enzymol. 100:468-500; and Zoller and Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13:8749-8764; Taylor et al. (1985) “The rapid generation of oligonucleotide-directed mutations at high frequency using phosphorothioate-modified DNA” Nucl. Acids Res. 13:8765-8787 (1985); Nakamaye and Eckstein (1986) “Inhibition of restriction endonuclease Nci I cleavage by phosphorothioate groups and its application to oligonucleotide-directed mutagenesis” Nucl. Acids Res. 14:9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16:803-814); mutagenesis using gapped duplex DNA (Kramer et al. (1984) “The gapped duplex DNA approach to oligonucleotide-directed mutation construction” Nucl. Acids Res. 12:9441-9456; Kramer and Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16:7207; and Fritz et al. (1988) “Oligonucleotide-directed construction of mutations: a gapped duplex DNA procedure without enzymatic reactions in vitro” Nucl. Acids Res. 16:6987-6999).

Additional suitable methods include point mismatch repair (Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13:4431-4443; and Carter (1987) “Improved oligonucleotide-directed mutagenesis using M13 vectors” Methods in Enzymol. 154:382-403), deletion mutagenesis (Eghtedarzadeh & Henikoff (1986) “Use of oligonucleotides to generate large deletions” Nucl. Acids Res. 14:5115), restriction-selection and restriction-selection and restriction-purification (Wells et al. (1986) “Importance of hydrogen-bond formation in stabilizing the transition state of subtilisin” Phil. Trans. R. Soc. Lond. A 317:415-423), mutagenesis by total gene synthesis (Nambiar et al. (1984) “Total synthesis and cloning of a gene coding for the ribonuclease S protein” Science 223:1299-1301; Sakamar and Khorana (1988) “Total synthesis and expression of a gene for the a-subunit of bovine rod outer segment guanine nucleotide-binding protein (transducin)” Nucl. Acids Res. 14:6361-6372; Wells et al. (1985) “Cassette mutagenesis: an efficient method for generation of multiple mutations at defined sites” Gene 34:315-323; and Grundström et al. (1985) “Oligonucleotide-directed mutagenesis by microscale ‘shot-gun’ gene synthesis” Nucl. Acids Res. 13:3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA 83:7177-7181). Additional details on many of the above methods can be found in Methods in Enzymology Volume 154, which also describes useful controls for trouble-shooting problems with various mutagenesis methods.

Additional details regarding artificially evolving enzymes can be found in the following U.S. patents, PCT publications, and EPO publications: U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/27230 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination.”

Certain U.S. applications provide additional details regarding various methods of artificially evolving enzymes, including “SHUFFLING OF CODON ALTERED GENES” by Patten et al. filed Sep. 28, 1999, (U.S. Ser. No. 09/407,800); “EVOLUTION OF WHOLE CELLS AND ORGANISMS BY RECURSIVE SEQUENCE RECOMBINATION” by del Cardayre et al., filed Jul. 15, 1998 (U.S. Ser. No. 09/166,188), and Jul. 15, 1999 (U.S. Ser. No. 09/354,922); “OLIGONUCLEOTIDE MEDIATED NUCLEIC ACID RECOMBINATION” by Crameri et al., filed Sep. 28, 1999 (U.S. Ser. No. 09/408,392), and “OLIGONUCLEOTIDE MEDIATED NUCLEIC ACID RECOMBINATION” by Crameri et al., filed Jan. 18, 2000 (PCT/US00/01203); “USE OF CODON-VARIED OLIGONUCLEOTIDE SYNTHESIS FOR SYNTHETIC SHUFFLING” by Welch et al., filed Sep. 28, 1999 (U.S. SER. NO. 09/408,393); “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. SER. NO. 09/618,579); “METHODS OF POPULATING DATA STRUCTURES FOR USE IN EVOLUTIONARY SIMULATIONS” by Selifonov and Stemmer, filed Jan. 18, 2000 (PCT/US00/01138); and “SINGLE-STRANDED NUCLEIC ACID TEMPLATE-MEDIATED RECOMBINATION AND NUCLEIC ACID FRAGMENT ISOLATION” by Affholter, filed Sep. 6, 2000 (U.S. SER. NO. 09/656,549).

The following exemplify some of the different types of preferred formats for artificially evolving enzymes in the context of the present invention, including, e.g., certain recombination based formats.

Nucleic acids can be recombined in vitro by any of a variety of techniques discussed in the references above, including, e.g., DNAse digestion of nucleic acids to be recombined followed by ligation and/or PCR reassembly of the nucleic acids. For example, sexual PCR mutagenesis can be used in which random (or pseudo random, or even non-random) fragmentation of the DNA molecule is followed by recombination, based on sequence similarity, between DNA molecules with different but related DNA sequences, in vitro, followed by fixation of the crossover by extension in a polymerase chain reaction. This process and many process variants are described in several of the references above including, e.g., in Stemmer (1994) Proc. Natl. Acad. Sci. USA 91:10747-10751.

Similarly, nucleic acids can be recursively recombined in vivo, e.g., by allowing recombination to occur between nucleic acids in cells. Many such in vivo recombination formats are set forth in the references noted above. Such formats optionally provide direct recombination between nucleic acids of interest, or provide recombination between vectors, viruses, plasmids, etc., comprising the nucleic acids of interest, as well as other formats. Details regarding such procedures are found in the references noted above.

Whole genome recombination methods can also be used in which whole genomes of cells or other organisms are recombined, optionally including spiking of the genomic recombination mixtures with desired library components (e.g., genes corresponding to the pathways of the present invention). These methods have many applications, including those in which the identity of a target gene is not known. Details regarding such methods are found, e.g., in WO 98/31837 by del Cardayre et al. “Evolution of Whole Cells and Organisms by Recursive Sequence Recombination;” and in, e.g., PCT/US99/15972 by del Cardayre et al., also entitled “Evolution of Whole Cells and Organisms by Recursive Sequence Recombination.”

Synthetic recombination methods can also be used, in which oligonucleotides corresponding to targets of interest are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by tri-nucleotide synthetic approaches. Details regarding such approaches are found in the references noted above, including, e.g., “OLIGONUCLEOTIDE MEDIATED NUCLEIC ACID RECOMBINATION” by Crameri et al., filed Sep. 28, 1999 (U.S. SER. NO. 09/408,392), and “OLIGONUCLEOTIDE MEDIATED NUCLEIC ACID RECOMBINATION” by Crameri et al., filed Jan. 18, 2000 (PCT/US00/01203); “USE OF CODON-VARIED OLIGONUCLEOTIDE SYNTHESIS FOR SYNTHETIC SHUFFLING” by Welch et al., filed Sep. 28, 1999 (U.S. SER. NO. 09/408,393); “METHODS FOR MAKING CHARACTER STRINGS, POLYNUCLEOTIDES AND POLYPEPTIDES HAVING DESIRED CHARACTERISTICS” by Selifonov et al., filed Jan. 18, 2000, (PCT/US00/01202); “METHODS OF POPULATING DATA STRUCTURES FOR USE IN EVOLUTIONARY SIMULATIONS” by Selifonov and Stemmer (PCT/US00/01138), filed Jan. 18, 2000; and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. SER. NO. 09/618,579).

In silico methods of recombination can be effected in which genetic algorithms are used in a computer to recombine sequence strings which correspond to homologous (or even non-homologous) nucleic acids. The resulting recombined sequence strings are optionally converted into nucleic acids by synthesis of nucleic acids that correspond to the recombined sequences, e.g., in concert with oligonucleotide synthesis/gene reassembly techniques. This approach can generate random, partially random or designed variants. Many details regarding in silico recombination, including the use of genetic algorithms, genetic operators and the like in computer systems, combined with generation of corresponding nucleic acids (and/or proteins), as well as combinations of designed nucleic acids and/or proteins (e.g., based on cross-over site selection) as well as designed, pseudo-random or random recombination methods are described in “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) “METHODS OF POPULATING DATA STRUCTURES FOR USE IN EVOLUTIONARY SIMULATIONS” by Selifonov and Stemmer (PCT/US00/01138), filed Jan. 18, 2000; and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. SER. NO. 09/618,579). Extensive details regarding in silico recombination methods are found in these applications. This methodology is generally applicable to the present invention in providing for recombination of, e.g., hydrolase or other encoding sequences in silico and/or the generation of corresponding nucleic acids or proteins.

Many methods of accessing natural diversity, e.g., by hybridization of diverse nucleic acids or nucleic acid fragments to single-stranded templates, followed by polymerization and/or ligation to regenerate full-length sequences, optionally followed by degradation of the templates and recovery of the resulting modified nucleic acids can be similarly used. In one method employing a single-stranded template, the fragment population derived from the genomic library or libraries is/are annealed with partial, or, often approximately full length ssDNA or RNA corresponding to the opposite strand. Assembly of complex chimeric genes from this population is then mediated by nuclease-base removal of non-hybridizing fragment ends, polymerization to fill gaps between such fragments and subsequent single stranded ligation. The parental polynucleotide strand can be removed by digestion (e.g., if RNA or uracil-containing), magnetic separation under denaturing conditions (if labeled in a manner conducive to such separation) and other available separation/purification methods. Alternatively, the parental strand is optionally co-purified with the chimeric strands and removed during subsequent screening and processing steps. Additional details regarding this approach are found, e.g., in “SINGLE-STRANDED NUCLEIC ACID TEMPLATE-MEDIATED RECOMBINATION AND NUCLEIC ACID FRAGMENT ISOLATION” by Affholter, U.S. SER. NO. 09/656,549, filed Sep. 6, 2000.

In another approach, single-stranded molecules are converted to double-stranded DNA (dsDNA) and the dsDNA molecules are bound to a solid support by ligand-mediated binding. After separation of unbound DNA, the selected DNA molecules are released from the support and introduced into a suitable host cell to generate a library enriched sequences that hybridize to the probe. A library produced in this manner provides a desirable substrate for further diversification using any of the procedures described herein.

Any of the preceding general recombination formats can be practiced in a reiterative fashion (e.g., one or more cycles of mutation/recombination or other diversity generation methods, optionally followed by one or more selection methods, such as the stereoselectivity screens described herein) to generate a more diverse set of recombinant nucleic acids.

Mutagenesis employing polynucleotide chain termination methods have also been proposed (see, e.g., U.S. Pat. No. 5,965,408, “Method of DNA reassembly by interrupting synthesis” to Short, and the references above), and can be applied to the present invention. In this approach, double stranded DNAs corresponding to one or more genes sharing regions of sequence similarity are combined and denatured, in the presence or absence of primers specific for the gene. The single stranded polynucleotides are then annealed and incubated in the presence of a polymerase and a chain terminating reagent (e.g., ultraviolet, gamma or X-ray irradiation; ethidium bromide or other intercalators; DNA binding proteins, such as single strand binding proteins, transcription activating factors, or histones; polycyclic aromatic hydrocarbons; trivalent chromium or a trivalent chromium salt; or abbreviated polymerization mediated by rapid thermocycling; and the like), resulting in the production of partial duplex molecules. The partial duplex molecules, e.g., containing partially extended chains, are then denatured and reannealed in subsequent rounds of replication or partial replication resulting in polynucleotides which share varying degrees of sequence similarity and which are diversified with respect to the starting population of DNA molecules. Optionally, the products, or partial pools of the products, can be amplified at one or more stages in the process. Polynucleotides produced by a chain termination method, such as described above, are suitable substrates for any other described recombination format.

Diversity also can be generated in nucleic acids or populations of nucleic acids using a recombinational procedure termed “incremental truncation for the creation of hybrid enzymes” (“ITCHY”) described in Ostermeier et al. (1999) “A combinatorial approach to hybrid enzymes independent of DNA homology” Nature Biotech 17:1205. This approach can be used to generate an initial a library of variants which can optionally serve as a substrate for one or more in vitro or in vivo recombination methods. See also, Ostermeier et al. (1999) “Combinatorial Protein Engineering by Incremental Truncation,” Proc. Natl. Acad. Sci. USA 96:3562-67; Ostermeier et al. (1999), “Incremental Truncation as a Strategy in the Engineering of Novel Biocatalysts,” Biological and Medicinal Chemistry 7:2139-44.

Mutational methods which result in the alteration of individual nucleotides or groups of contiguous or non-contiguous nucleotides can be favorably employed to introduce nucleotide diversity. Many mutagenesis methods are found in the above-cited references; additional details regarding mutagenesis methods can be found in following, which can also be applied to the present invention.

For example, error-prone PCR can be used to generate nucleic acid variants. Using this technique, PCR is performed under conditions where the copying fidelity of the DNA polymerase is low, such that a high rate of point mutations is obtained along the entire length of the PCR product. Examples of such techniques are found in the references above and, e.g., in Leung et al. (1989) Technique 1:11-15 and Caldwell et al. (1992) PCR Methods Applic. 2:28-33. Similarly, assembly PCR can be used, in a process which involves the assembly of a PCR product from a mixture of small DNA fragments. A large number of different PCR reactions can occur in parallel in the same reaction mixture, with the products of one reaction priming the products of another reaction.

Oligonucleotide directed mutagenesis can be used to introduce site-specific mutations in a nucleic acid sequence of interest. Examples of such techniques are found in the references above and, e.g., in Reidhaar-Olson et al. (1988) Science, 241:53-57. Similarly, cassette mutagenesis can be used in a process that replaces a small region of a double stranded DNA molecule with a synthetic oligonucleotide cassette that differs from the native sequence. The oligonucleotide can contain, e.g., completely and/or partially randomized native sequence(s).

Recursive ensemble mutagenesis is a process in which an algorithm for protein mutagenesis is used to produce diverse populations of phenotypically related mutants, members of which differ in amino acid sequence. This method uses a feedback mechanism to monitor successive rounds of combinatorial cassette mutagenesis. Examples of this approach are found in Arkin and Youvan (1992) Proc. Natl. Acad. Sci. USA 89:7811-7815.

Exponential ensemble mutagenesis can be used for generating combinatorial libraries with a high percentage of unique and functional mutants. Small groups of residues in a sequence of interest are randomized in parallel to identify, at each altered position, amino acids which lead to functional proteins. Examples of such procedures are found in Delegrave and Youvan (1993) Biotechnology Research 11:1548-1552.

In vivo mutagenesis can be used to generate random mutations in any cloned DNA of interest by propagating the DNA, e.g., in a strain of E. coli that carries mutations in one or more of the DNA repair pathways. These “mutator” strains have a higher random mutation rate than that of a wild-type parent. Propagating the DNA in one of these strains will eventually generate random mutations within the DNA. Such procedures are described in the references noted above.

Other procedures for introducing diversity into a genome, e.g., a bacterial, fungal, animal or plant genome can be used in conjunction with the above described and/or referenced methods. For example, in addition to the methods above, techniques have been proposed which produce nucleic acid multimers suitable for transformation into a variety of species (see, e.g., Schellenberger, U.S. Pat. No. 5,756,316 and the references above). Transformation of a suitable host with such multimers, consisting of genes that are divergent with respect to one another, (e.g., derived from natural diversity or through application of site directed mutagenesis, error prone PCR, passage through mutagenic bacterial strains, and the like), provides a source of nucleic acid diversity for DNA diversification, e.g., by an in vivo recombination process as indicated above.

Alternatively, a multiplicity of monomeric polynucleotides sharing regions of partial sequence similarity can be transformed into a host species and recombined in vivo by the host cell. Subsequent rounds of cell division can be used to generate libraries, members of which, include a single, homogenous population, or pool of monomeric polynucleotides. Alternatively, the monomeric nucleic acid can be recovered by standard techniques, e.g., PCR and/or cloning, and recombined in any of the recombination formats, including recursive recombination formats, described above.

Methods for generating multispecies expression libraries have been described (in addition to the reference noted above, see, e.g., Peterson et al. (1998) U.S. Pat. No. 5,783,431 “METHODS FOR GENERATING AND SCREENING NOVEL METABOLIC PATHWAYS,” and Thompson, et al. (1998) U.S. Pat. No. 5,824,485 METHODS FOR GENERATING AND SCREENING NOVEL METABOLIC PATHWAYS) and their use to identify protein activities of interest has been proposed (In addition to the references noted above, see, Short (1999) U.S. Pat. No. 5,958,672 “PROTEIN ACTIVITY SCREENING OF CLONES HAVING DNA FROM UNCULTIVATED MICROORGANISMS”). Multispecies expression libraries include, in general, libraries comprising cDNA or genomic sequences from a plurality of species or strains, operably linked to appropriate regulatory sequences, in an expression cassette. The cDNA and/or genomic sequences are optionally randomly ligated to further enhance diversity. The vector can be a shuttle vector suitable for transformation and expression in more than one species of host organism, e.g., bacterial species, eukaryotic cells. In some cases, the library is biased by preselecting sequences which encode a protein of interest, or which hybridize to a nucleic acid of interest. Any such libraries can be provided as substrates for any of the methods described herein.

The above described procedures have been largely directed to increasing nucleic acid and/or encoded protein diversity. However, in many cases, not all of the diversity is useful, e.g., functional, and contributes merely to increasing the background of variants that must be screened or selected to identify the few favorable variants. In some applications, it is desirable to preselect or prescreen libraries (e.g., an amplified library, a genomic library, a cDNA library, a normalized library, etc.) or other substrate nucleic acids prior to diversification, e.g., by recombination-based mutagenesis procedures, or to otherwise bias the substrates towards nucleic acids that encode functional products. For example, in the case of antibody engineering, it is possible to bias the diversity generating process toward antibodies with functional antigen binding sites by taking advantage of in vivo recombination events prior to manipulation by any of the described methods. For example, recombined CDRs derived from B cell cDNA libraries can be amplified and assembled into framework regions (e.g., Jirholt et al. (1998) “Exploiting sequence space: shuffling in vivo formed complementarity determining regions into a master framework” Gene 215:471) prior to diversifying according to any of the methods described herein.

Libraries can be biased towards nucleic acids which encode proteins with desirable enzyme activities, such as the ability to stereoselectively catalyze a given reaction. For example, after identifying a clone from a library which exhibits a specified activity, the clone can be mutagenized using any known method for introducing DNA alterations. A library comprising the mutagenized homologues is then screened for a desired activity, which can be the same as or different from the initially specified activity. An example of such a procedure is proposed in Short (1999) U.S. Pat. No. 5,939,250 for “PRODUCTION OF ENZYMES HAVING DESIRED ACTIVITIES BY MUTAGENESIS.” Desired activities can be identified by any method known in the art. For example, WO 99/10539 proposes that gene libraries can be screened by combining extracts from the gene library with components obtained from metabolically rich cells and identifying combinations which exhibit the desired activity. It has also been proposed (e.g., WO 98/58085) that clones with desired activities can be identified by inserting bioactive substrates into samples of the library, and detecting bioactive fluorescence corresponding to the product of a desired activity using a fluorescent analyzer, e.g., a flow cytometry device, a CCD, a fluorometer, or a spectrophotometer.

Libraries can also be biased towards nucleic acids which have specified characteristics, e.g., hybridization to a selected nucleic acid probe. For example, application WO 99/10539 proposes that polynucleotides encoding a desired activity (e.g., an enzymatic activity, for example: a lipase, an esterase, a protease, a glycosidase, a glycosyl transferase, a phosphatase, a kinase, an oxygenase, a peroxidase, a hydrolase, a hydratase, a nitrilase, a transaminase, an amidase or an acylase) can be identified from among genomic DNA sequences in the following manner. Single stranded DNA molecules from a population of genomic DNA are hybridized to a ligand-conjugated probe. The genomic DNA can be derived from either a cultivated or uncultivated microorganism, or from an environmental sample. Alternatively, the genomic DNA can be derived from a multicellular organism, or a tissue derived therefrom. Second strand synthesis can be conducted directly from the hybridization probe used in the capture, with or without prior release from the capture medium or by a wide variety of other strategies known in the art. Alternatively, the isolated single-stranded genomic DNA population can be fragmented without further cloning and used directly in, e.g., a recombination-based approach, that employs a single-stranded template, as described above.

“Non-Stochastic” methods of generating nucleic acids and polypeptides are alleged in Short “Non-Stochastic Generation of Genetic Vaccines and Enzymes” WO 00/46344. These methods, including proposed non-stochastic polynucleotide reassembly and site-saturation mutagenesis methods can be applied to the present invention as well. Random or semi-random mutagenesis using doped or degenerate oligonucleotides is also described in, e.g., Arkin and Youvan (1992) “Optimizing nucleotide mixtures to encode specific subsets of amino acids for semi-random mutagenesis” Biotechnology 10:297-300; Reidhaar-Olson et al. (1991) “Random mutagenesis of protein sequences using oligonucleotide cassettes” Methods Enzymol. 208:564-86; Lim and Sauer (1991) “The role of internal packing interactions in determining the structure and stability of a protein” J. Mol. Biol. 219:359-76; Breyer and Sauer (1989) “Mutational analysis of the fine specificity of binding of monoclonal antibody 51F to lambda repressor” J. Biol. Chem. 264:13355-60); and “Walk-Through Mutagenesis” (Crea, R; U.S. Pat. Nos. 5,830,650 and 5,798,208, and EP Patent 0527809 B1.

It will readily be appreciated that any of the above described techniques suitable for enriching a library prior to diversification are optionally also used to screen the products, or libraries of products, produced by the diversity generating methods.

Kits for mutagenesis, library construction and other diversity generation methods are also commercially available. For example, kits are available from, e.g., Stratagene (e.g., QuickChange™ site-directed mutagenesis kit; and Chameleon™ double-stranded, site-directed mutagenesis kit), Bio/Can Scientific, Bio-Rad (e.g., using the Kunkel method described above), Boehringer Mannheim Corp., Clonetech Laboratories, DNA Technologies, Epicentre Technologies (e.g., 5 prime 3 prime kit); Genpak Inc, Lemargo Inc, Life Technologies (Gibco BRL), New England Biolabs, Pharmacia Biotech, Promega Corp., Quantum Biotechnologies, Amersham International plc (e.g., using the Eckstein method above), and Anglian Biotechnology Ltd (e.g., using the Carter/Winter method above).

The above references provide many mutational formats, including recombination, recursive recombination, recursive mutation and combinations or recombination with other forms of mutagenesis, as well as many modifications of these formats. Regardless of the diversity generation format that is used, the nucleic acids of the invention can be recombined (with each other, or with related (or even unrelated) sequences) to produce a diverse set of recombinant nucleic acids, including, e.g., sets of homologous nucleic acids, as well as corresponding polypeptides.

The nucleic acids produced by the methods described above are typically cloned into cells for expression and subsequent stereoselectivity screening (or used in in vitro transcription reactions to make products which are screened). General texts which describe molecular biological techniques useful herein, including mutagenesis, library construction, cell culture, and the like include Berger and Kimmel, Guide to Molecular Cloning Techniques, Methods in Enzymology volume 152 Academic Press, Inc., San Diego, Calif. (Berger); Sambrook et al., Molecular Cloning - A Laboratory Manual (2nd Ed.), Vol. 1-3, Cold Spring Harbor Laboratory, Cold Spring Harbor, N.Y., 1989 (Sambrook) and Current Protocols in Molecular Biology, F. M. Ausubel et al., eds., Current Protocols, a joint venture between Greene Publishing Associates, Inc. and John Wiley & Sons, Inc., New York (supplemented through 1999) (Ausubel)). Methods of transducing cells, including plant and animal cells, with nucleic acids are generally available, as are methods of expressing proteins encoded by such nucleic acids. In addition to Berger, Ausubel and Sambrook, useful general references for culture of animal cells include Freshney (Culture of Animal Cells, a Manual of Basic Technique, third edition Wiley-Liss, New York (1994)) and the references cited therein, Humason (Animal Tissue Techniques, fourth edition W. H. Freeman and Company (1979)) and Ricciardelli, et al., In Vitro Cell Dev. Biol. 25:1016-1024 (1989). References for plant cell cloning, culture and regeneration include Payne et al. (1992) Plant Cell and Tissue Culture in Liquid Systems John Wiley & Sons, Inc. New York, N.Y. (Payne); and Gamborg and Phillips (eds) (1995) Plant Cell, Tissue and Organ Culture; Fundamental Methods Springer Lab Manual, Springer-Verlag (Berlin Heidelberg N.Y.) (Gamborg). A variety of Cell culture media are described in Atlas and Parks (eds) The Handbook of Microbiological Media (1993) CRC Press, Boca Raton, Fla. (Atlas). Additional information for plant cell culture is found in available commercial literature such as the Life Science Research Cell Culture Catalogue (1998) from Sigma-Aldrich, Inc (St Louis, Mo.) (Sigma-LSRCCC) and, e.g., the Plant Culture Catalogue and supplement (1997) also from Sigma-Aldrich, Inc (St Louis, Mo.) (Sigma-PCCS).

Examples of techniques sufficient to direct persons of skill through in vitro amplification methods, useful e.g., for amplifying oligonucleotide shuffled nucleic acids including the polymerase chain reaction (PCR), the ligase chain reaction (LCR), Qβ-replicase amplification, and other RNA polymerase mediated techniques (e.g., NASBA). These techniques are found in Berger, Sambrook, and Ausubel, id., as well as in Mullis et al., (1987) U.S. Pat. No. 4,683,202; PCR Protocols A Guide to Methods and Applications (Innis et al. eds) Academic Press Inc. San Diego, Calif. (1990) (Innis); Arnheim and Levinson (Oct. 1, 1990) C&EN 36-47; The Journal Of NIH Research (1991) 3:81-94; Kwoh et al. (1989) Proc. Natl. Acad. Sci. USA 86, 1173; Guatelli et al. (1990) Proc. Natl. Acad. Sci. USA 87:1874; Lomell et al. (1989) J. Clin. Chem 35, 1826; Landegren et al., (1988) Science 241:1077-1080; Van Brunt (1990) Biotechnology 8:291-294; Wu and Wallace, (1989) Gene 4:560; Barringer et al. (1990) Gene 89:117, and Sooknanan and Malek (1995) Biotechnology 13:563-564. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369:684-685 and the references therein, in which PCR amplicons of up to 40 kb are generated.

G. Kits

The present invention also provides kits packaged to include many, if not all, of the necessary reagents, e.g., libraries, substrate molecules, or the like for performing any of the enzyme screens described herein. Such kits also optionally include appropriate containers and instructions for using the systems described herein as well as necessary reagents, and in cases where reagents are not predisposed in elements of the systems, with appropriate instructions for introducing the reagents into the library storage or preparation medium (e.g., a microtiter dish or duplicate dish) or mass spectrometer of the system. Such kits typically include a preparation plate with necessary reagents, e.g., a library, substrate molecules, or the like predisposed in the wells or separately packaged. Generally, such reagents are provided in a stabilized form, so as to prevent degradation or other loss during prolonged storage, e.g., from leakage. A number of stabilizing processes are widely used for reagents that are to be stored, such as the inclusion of chemical stabilizers (i.e., enzymatic inhibitors, microcides/bacteriostats, anticoagulants), the physical stabilization of the material, e.g., through immobilization on a solid support, entrapment in a matrix (i.e., a gel), lyophilization, or the like.

### EXAMPLE

I. Substrate Synthesis

All materials were purchased from Sigma or Aldrich unless noted. Nerol butyrate was prepared by from nerol and butyryl chloride in methylene chloride/pyridine. Geranyl deuterobutyrate was prepared from geraniol and deuterobutyric acid (Isotec) using DCC coupling in methylene chloride. Both compounds were purified by flash chromatography (ether/hexanes) and gave satisfactory analysis by mass spectrometry and NMR.

II. Library Pre-Selection and Enzyme Preparation

An artificially evolved lipase library was prepared by shuffling, using methods described in WO 97/20078. Transformants were robotically picked to 386-well microtiter plates containing 70 μL growth medium (2× YT, 0.5% glucose to suppress induction, 30 μg/ml chloramphenicol) and grown 12-20 hours at 37° C., 300-rpm shaking speed in a Kuhner incubator. The cultures were then gridded via a Q-bot robot (Genetix, UK) to inducing agar (2× YT, 1.5% agar, 1 mM IPTG, 30 μg/ml chloramphenicol) in 22 cm ×22 cm bioassay trays using 0.25 mm pins, and incubated at 30° C. for 16-20 hours. The colonies were then overlaid with substrate (1% nerol acetate or geraniol acetate) in 150 mL of 1.5% agar containing 2 mM Hepes, pH 7.4, and 1% Triton X-100 that had been heated to 45° C. The reaction was allowed to proceed at room temperature for 5 to 20 hours, until clearing zones around active colonies were visible. The trays were imaged against a black background with an Alpha Innotech Fluorchem imaging system, and the images were analyzed using Phoretix Array image analysis software. Active clones were identified based upon the intensity of the corresponding clearing zone, and transferred (5 μL) from the master 384-well plates to rows 1-7 of 96 well microtiter plates containing 200 μL growth medium. The final row of the 96-well plate was spiked with 5 μL cultures transformed with a plasmid that did not contain an active lipase as a negative background control. The cultures were grown overnight at 37° C. at 200-230 rpm shaking speed in a Kuhner incubator. The following day, 10 μL of each culture was dispensed into 200 μL inducing media (2× YT, 1 mM IPTG, 30 μg/ml chloramphenicol) in a second 96-well plate. The cultures were induced for 16-20 hours at 30° C., 200 rpm in a Kuhner incubator. The cells were then pelleted by centrifugation and the lipase-containing supernatant assayed as described below.

III. Reactions, Mass Spectrometrical Analysis, and Results

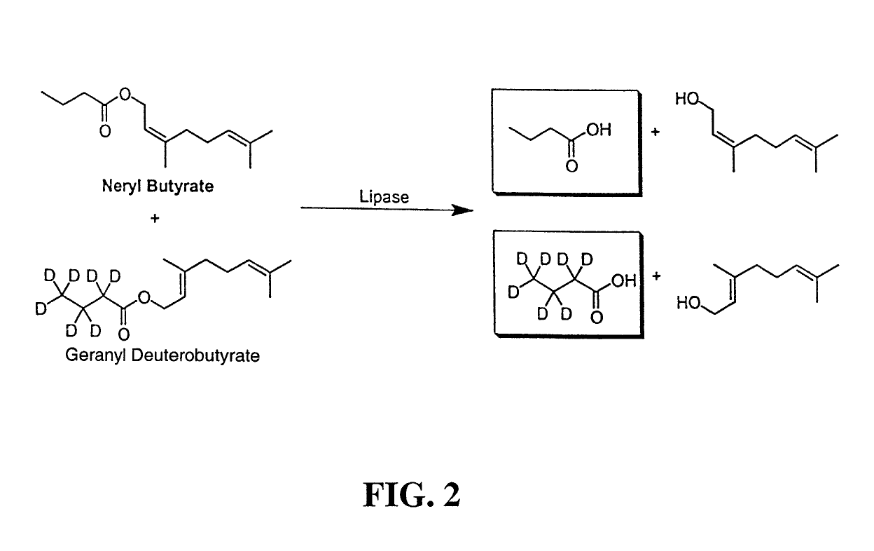

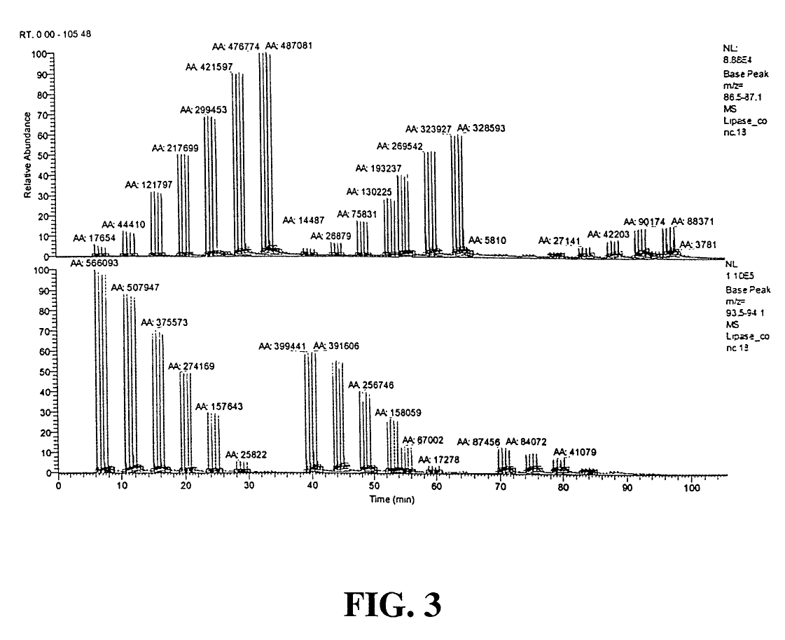

10 μL of cell supernatant was added to 90 μL reaction mix that contained 2.78 mM neryl butyrate, 2.78 mM geraniol deuterobutyrate, and 1 mM morpholine acetate, pH 7.4, in a 96-well plate. FIG. 2 schematically depicts acyl cleavage reactions catalyzed by the lipases used in these screens. The plates were sealed with plastic tape and shaken on a MicroMix (Diagnostics Products Corporation) set to mix at amplitude 4, form 20. After 8 hours, 10 μL of this reaction mix was added to 90 μL 40:50 H₂O:MeOH. The final row of the plate was spiked with known concentrations of butyrate and deuterobutyrate (0-50 uM) to provide calibration curves. The plates were sealed (MicroLiter Analytical polypropylene & aluminum foil film) and analyzed by LC/MS for butyrate and deuterobutyrate concentrations. Clones showing desired specificity were then reconfirmed by GC/MS. FIG. 3 provides data graphs showing the quantification of different ratios of butyrate (top graph) and deuterobutyrate (bottom graph) simultaneously by mass spectrometry.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above may be used in various combinations. All publications, patents, patent applications, or other documents cited in this application are incorporated by reference in their entirety for all purposes to the same extent as if each individual publication, patent, patent application, or other document were individually indicated to be incorporated by reference for all purposes.

What is claimed is:

1. A method of screening for enzyme stereoselectivity, comprising:
(a) providing a plurality of substrate molecules,
wherein the plurality comprises two or more substrate molecule types,
wherein at least one of the substrate molecule types has one or more leaving groups,
wherein at least one of the leaving groups is isotopically labeled;
(b) contacting at least one enzyme with the plurality of substrate molecules,
wherein the enzyme converts one or more of the substrate molecules to two or more products,
(c) quantifying the two or more products mass spectrometrically, wherein at least one of the quantified products comprises the isotopically labeled leaving group,
thereby screening for enzyme stereoselectivity.

2. The method of claim 1, wherein each substrate molecule type comprises a leaving group.

3. The method of claim 1, wherein the enzyme is a hydrolase.

4. The method of claim 1, wherein at least one of the products comprises three or more carbon atoms.

5. The method of claim 4, wherein the isotopically labeled leaving group comprises three or more carbon atoms.

6. The method of claim 4, wherein at least one of the products comprises four or more carbon atoms.

7. The method of claim 6, wherein the isotopically labeled leaving group comprises four or more carbon atoms.

8. The method of claim 1, wherein the isotopic label is selected from the group consisting of ²H, ³H, ⁷Li, ¹³C, ¹⁴C, ¹¹B, ¹⁹F, ³¹P, ³²P, ¹⁵N, ¹⁷O, and ¹⁸O.

9. The method of claim 8, wherein the isotopic label is ²H.

10. The method of claim 1, wherein the products are quantified after less than about 10% conversion of substrate(s) to product(s).

11. The method of claim 10, wherein the products are quantified after less than about 5% conversion of substrate(s) to product(s).

12. The method of claim 11, wherein the products are quantified after less than about 3% conversion of substrate(s) to product(s).

13. The method of claim 1, wherein the plurality of substrate molecules comprises pseudo-enantiomers.

14. The method of claim 1, wherein the plurality of substrate molecules comprises pseudo-diastereomers.

15. The method of claim 1, wherein the plurality of substrate molecules comprises pseudo-meso compounds.

16. The method of claim 1, wherein the plurality of substrate molecules comprises esters.

17. The method of claim 1, wherein the plurality of substrate molecules comprises a pseudo-racemate.

18. The method of claim 1, wherein step (b) is conducted on a cell growth plate.

19. The method of claim 1, wherein the at least one enzyme is an enzyme library.

20. The method of claim 1, further comprising separating the products prior to application of mass spectrometry by a method selected from the group consisting of liquid chromatography, gas chromatography, and capillary zone electrophoresis.

21. A method of screening for enzyme stereoselectivity, comprising:
(a) providing a pseudo-meso substrate molecule comprising at least one isotopically labeled leaving group;
(b) contacting at least one enzyme with the pseudo-meso substrate molecule,
wherein the enzyme converts the pseudo-meso substrate molecule to two or more products;
(c) quantifying the two or more products mass spectrometrically, wherein at least one of the quantified products comprises the isotopically labeled leaving group,
thereby screening for enzyme stereoselectivity.

22. The method of claim 21, wherein the enzyme is a hydrolase.

23. The method of claim 21, wherein at least one of the products comprises three or more carbon atoms.

24. The method of claim 23, wherein the isotopically labeled leaving group comprises three or more carbon atoms.

25. The method of claim 23, wherein at least one of the products comprises four or more carbon atoms.

26. The method of claim 25, wherein the isotopically labeled leaving group comprises four or more carbon atoms.

27. The method of claim 26, wherein the products are quantified after less than about 10% conversion of substrate to products.

28. The method of claim 27, wherein the products are quantified after less than about 5% conversion of substrate to products.

30. The method of claim 21, further comprising separating the products prior to application of mass spectrometry by a method selected from the group consisting of liquid chromatography, gas chromatography, and capillary zone electrophoresis.
